SEQUENCE LISTING (5 sequences)

SEQ ID NO: 1 - protein, 220 aa, Homo sapiens
MLRLLLALNLFPSIQVTGNKILVKQSPMLVAYDNAVNLSCKYSYNLFSREFRASLHKGLDSAVEVCVVYGNYSQQLQVYSKTGFNCDGKLGNESVTFYLQNLYVNQTDIYFCKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 2 - protein, 135 aa, Homo sapiens
PSIQVTGNKILVKQSPMLVAYDNAVNLSCKYSYNLFSREFRASLHKGLDSAVEVCVVYGNYSQQLQVYSKTGFNCDGKLGNESVTFYLQNLYVNQTDIYFCKIEVMYPPPYLDNEKSNGTIIHVKGKHLCPSPLF

SEQ ID NO: 3 - DNA, 3804 bp, Homo sapiens
taaagtcatcaaaacaacgttatatcctgtgtgaaatgctgcagtcaggatgccttgtggtttgagtgccttgatcatgtgccctaaggggatggtggcggtggtggtggccgtggatgacggagactctcaggccttggcaggtgcgtctttcagttcccctcacacttcgggttcctcggggaggaggggctggaaccctagcccatcgtcaggacaaagatgctcaggctgctcttggctctcaacttattcccttcaattcaagtaacaggaaacaagattttggtgaagcagtcgcccatgcttgtagcgtacgacaatgcggtcaaccttagctgcaagtattcctacaatctcttctcaagggagttccgggcatcccttcacaaaggactggatagtgctgtggaagtctgtgttgtatatgggaattactcccagcagcttcaggtttactcaaaaacggggttcaactgtgatgggaaattgggcaatgaatcagtgacattctacctccagaatttgtatgttaaccaaacagatatttacttctgcaaaattgaagttatgtatcctcctccttacctagacaatgagaagagcaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctcctgacacggacgcctatccagaagccagccggctggcagcccccatctgctcaatatcactgctctggataggaaatgaccgccatctccagccggccacctcaggcccctgttgggccaccaatgccaatttttctcgagtgactagaccaaatatcaagatcattttgagactctgaaatgaagtaaaagagatttcctgtgacaggccaagtcttacagtgccatggcccacattccaacttaccatgtacttagtgacttgactgagaagttagggtagaaaacaaaaagggagtggattctgggagcctcttccctttctcactcacctgcacatctcagtcaagcaaagtgtggtatccacagacattttagttgcagaagaaaggctaggaaatcattccttttggttaaatgggtgtttaatcttttggttagtgggttaaacggggtaagttagagtagggggagggataggaagacatatttaaaaaccattaaaacactgtctcccactcatgaaatgagccacgtagttcctatttaatgctgttttcctttagtttagaaatacatagacattgtcttttatgaattctgatcatatttagtcattttgaccaaatgagggatttggtcaaatgagggattccctcaaagcaatatcaggtaaaccaagttgctttcctcactccctgtcatgagacttcagtgttaatgttcacaatatactttcgaaagaataaaatagttctcctacatgaagaaagaatatgtcaggaaataaggtcactttatgtcaaaattatttgagtactatgggacctggcgcagtggctcatgcttgtaatcccagcactttgggaggccgaggtgggcagatcacttgagatcaggaccagcctggtcaagatggtgaaactccgtctgtactaaaaatacaaaatttagcttggcctggtggcaggcacctgtaatcccagctgcccaggaggctgaggcatgagaatcgcttgaacctggcaggcggaggttgcagtgagccgagatagtgccacagctctccagcctgggcgacagagtgagactccatctcaaacaacaacaacaacaacaacaacaacaacaaaccacaaaattatttgagtactgtgaaggattatttgtctaacagttcattccaatcagaccaggtaggagctttcctgtttcatatgtttcagggttgcacagttggtctctttaatgtcggtgtggagatccaaagtgggttgtggaaagagcgtccataggagaagtgagaatactgtgaaaagggatgttagcattcattagagtatgaggatgagtcccaagaaggttctttggaaggaggacgaatagaatggagtaatgaaattcttgccatgtgctgaggagatagccagcattaggtgacaatcttccagaagtggtcaggcagaaggtgccctggtgagagctcctttacagggactttatgtggtttagggctcagagctccaaaactctgggctcagctgctcctgtaccttggaggtccattcacatgggaaagtattttggaatgtgtcttttgaagagagcatcagagttcttaagggactgggtaaggcctgaccctgaaatgaccatggatatttttctacctacagtttgagtcaactagaatatgcctggggaccttgaagaatgcccttcagtggccctcaccatttgttcatgcttcagttaattcaggtgttgaaggagcttaggttttagaggcacgtagacttggttcaagtctcgttagtagttgaatagcctcaggcaagtcactgcccacctaagatgatggttcttcaactataaatggagataatggttacaaatgtctcttcctatagtataatctccataagggcatggcccaagtctgtctttgactctgcctatccctgacgtttagtagcatgcccgacatacaatgttagctattggtattattgccatatagataaattatgtataaaaattaaactgggcaatagcctaagaaggggggaatattgtaacacaaatttaaacccactacgcagggatgaggtgctataatatgaggaccttttaacttccatcattttcctgtttcttgaaatagtttatcttgtaatgaaatataaggcacctcccacttttatgtatagaaagaggtcttttaatttttttttaatgtgagaaggaagggaggagtaggaatcttgagattccatatcgaaaatactgtactttggttgatttttaagtgggcttccattccatggatttaatcagtcccaagaagatcaaactcagcagtacttgggtgctgaagaactgttggatttaccctggcacgtgtgccacttgcccagcttcttgggcacacagagttcttcaatccaagttatcagattgtatttgaaaatgacagagctggagagttttttgaaatggcagtggcaaataaataaatacttttttttaaatggaaagacttgatctatggtaataaatgattttgttttctgactggaaaaataggcctactaaagatgaatcacacttgagatgtttcttactcactctgcacagaaacaaagaagaaatgttatacagggaagtccgttttcactattagtatgaaccaagaaatggttcaaaaacagtggtaggagcaatgctttcatagtttcagatatggtagttatgaagaaaacaatgtcatttgctgctattattgtaagagtcttataattaatggtactcctataatttttgattgtgagctcacctatttgggttaagcatgccaatttaaagagaccaagtgtatgtacattatgttctacatattcagtgataaaattactaaactact

SEQ ID NO: 4 - DNA, 33 bp, Artificial Sequence
  Description of Artificial Sequence  Synthetic oligonucleotide
aaagaattcccttcaattcaagtaacaggaaac

SEQ ID NO: 5 - DNA, 30 bp, Artificial Sequence
  Description of Artificial Sequence  Synthetic oligonucleotide
aaacccgggaaataggggacttggacaaag

(19) United States
(12) Patent Application Publication
(10) Pub. No.: US 2009/0117582 A1
(43) Pub. Date: May 7, 2009

(54) Diagnosis of Allergic Complaints, Atopic Diseases and/or Auto-Immune Diseases by the Identification of Antibodies Against CD28 in Human Serum

(75) Inventors: Karsten Neuber, Hamburg (DE); Birgit Mahnss, Elmshorn (DE); Christian Hubner, Hamburg (DE)

Correspondence Address: ARNOLD & PORTER LLP;ATTN: IP DOCKETING DEPT., 555 TWELFTH STREET, N.W., WASHINGTON, DC, 20004-1206, US

(73) Assignee: Universitatskilinikum Hamburg-Eppenford, Hamburg (DE)

(21) Appl. No.: 11/815,734

(22) PCT Filed: Feb. 3, 2006
(86) PCT No.: PCT/EP06/00947

§ 371 Date: May 15, 2008

(30) Foreign Application Priority Data
Feb. 7, 2005 (DE) 10 2005 006 217.2

(51) Int. Cl. G01N 33/53

(52) U.S. Cl. 435/7.1; 436/501; 435/7.9; 435/7.92

(57) ABSTRACT

The invention relates to a method for diagnosing allergic complaints, atopic diseases and/or auto-immune diseases, according to which a sample from a patient is analysed for the presence of anti-CD28 auto-antibodies by bringing said sample into contact with CD28. If auto-antibodies bond to the CD28, this indicates the presence of an allergic complaint, atopic disease and/or auto-immune disease. The invention also relates to the use of CD28 for diagnosing said diseases and to a kit that is designed for this purpose, comprising CD28 and marked anti-immunoglobulin antibodies.

The present invention relates to a method for the diagnosis of allergic diseases, atopic diseases and/or autoimmune diseases, in which a sample from a patient is analysed for the presence of anti-CD28 autoantibodies by bringing the sample into contact with CD28, wherein binding of the autoantibodies to CD28 indicates the presence of an allergic disease, atopic disease and/or autoimmune disease. The invention further relates to the use of CD28 for the diagnosis of said diseases and a kit devised for this purpose, which comprises CD28 and labeled anti-immunoglobulin antibodies.

The adaptive immune response is an important component of the body's system for defense to infections and is therefore essential for the maintenance of health.

However, adaptive immune responses are also sometimes triggered by antigens that have nothing to do with infectious organisms. Such inappropriate immune responses can lead to serious diseases, including allergies, atopic diseases or autoimmunity.

An autoimmune disease is a specific adaptive immune reaction to endogenous antigens. We do not know what triggers autoimmune reactions, but it is extremely likely that both environmental and hereditary factors play a role. Autoimmune diseases usually lead to long-term tissue damage, as the cells that express the autoantigens that are recognized by the immune system may be destroyed. This probably involves mainly cytotoxic T-cells and excessive activation of macrophages. Harmful antibody reactions may also play a role.

Allergies are immune system-mediated reactions to a foreign substance that is normally harmless. Allergic reactions do not occur at the very first contact with the allergen. The first adaptive immune reaction takes time and as a rule goes unnoticed. However, as soon as antibodies or T-cells directed against the antigen are induced, each new contact with this antigen produces symptoms.

There are various types of tissue damage caused by immune reactions. In the case of allergies, fast allergic reactions mediated by IgE antibodies, so-called immediate-type hypersensitivity, atopic allergy or atopy, play a decisive role. In the case of delayed-type hypersensitivity, T-cell responses are the cause, and they do not reach their maximum until after a day or two.

The prevalence of allergic and atopic diseases has increased sharply in recent decades. More than 20% of the population suffer from allergies of the immediate type.

A possible explanation for the increase e.g. in atopic dermatitis is the so-called hygiene hypothesis, which assumes that atopic diseases can be prevented by infections in childhood. This theory is supported by known risk factors for the development of atopic diseases, such as small families or living in centers of population. Immunological indications also support the hygiene hypothesis.

The current pathophysiological concept of atopy is based on the assumption that allergen-specific T-lymphocytes of the Th2 type, which secrete certain cytokines, primarily interleukin (IL)-4, IL-5, IL-10, IL-13 and granulocyte-macrophage colony stimulating factor (GM-CSF), dominate the immune reaction, whereas the Th1 lymphocytes that produce for example interferon (IFN)-γ are less active (Jujo et al., J Allergy Clin Immunol 1992, 90: 323-331). Cytokines such as IL-4, IL-5 and IL-13 are mainly responsible for eosinophilia and increased production of antibodies of the IgE isotype in patients with atopic disorders (Punnonen et al., Proc Natl Acad Sci USA 1993, 90: 3730-3734). In patients with atopic disorders there is thus found to be a general shift of the equilibrium of the immune system from Th1 to Th2 responses. Generally, Th1 responses are more likely to be induced by infections, for example bacterial infections, whereas Th2 responses are triggered for example as reactions to attack, e.g. by parasitic worms.

In many allergic diseases the causative allergen is known or can be determined by allergy tests. So-called skin prick tests are mainly used for this, especially in the case of allergies of the immediate type (Dreborg, J Am Acad Dermatol. 1989, 21:820-821). With these tests it is possible, for example, for the causative agent of hayfever to be determined with great certainty in a short time. In this case identification of the allergen often makes it possible to avoid or reduce exposure to the allergen, and sometimes so-called specific immunotherapy is also possible, and can result in desensitization of the patient.

This is accompanied by a decrease in the concentration of specific IgEs and an increase in the concentration of specific IgG4s (Reid et al., J. Allergy Clin. Immunol. 78: 590-600, 1986), and a decrease in the number of mast cells and eosinophils and reduced secretion of mediators (Varney et al., J. Clin. Invest. 92: 644-651, 1993). Induction of energy of T-cells and a shift of the cytokine spectrum toward production of IL-10 and Th1-cytokines also seems to play a role (Akdis and Blaser, Allergy 55: 522-530, 2000, Akdis et al., J. Clin. Invest. 102: 98-106, 1998).

However, for many diseases in the allergy classification it is not easy to establish the causative agents. With atopic dermatitis or asthma for example, the diagnosis is generally based mainly on the symptoms, and it is difficult to identify individual causative agents. Treatment is also generally directed at alleviation of the symptoms.

Early therapy can, however, be decisive for counteracting exacerbation of the condition in the longer term. For example, in sensitized children with atopic dermatitis the administration of a modern antihistamine can prevent exacerbation, or early antiinflammatory therapy with inhaled steroids can greatly improve the quality of life of children with bronchial asthma.

Atopy also denotes an inherited tendency to suffer from one or more of the following atopic diseases: atopic bronchial asthma, allergic rhinoconjunctivitis (hayfever) or atopic dermatitis (atopic eczema). For the diagnosis of atopy, there is no single clinical sign or particular laboratory test result—the diagnosis is generally based on a combination of clinical features and the patient's records and family medical history.

When taking the medical history, particular attention is paid to the presence of eczema, allergic asthma and allergic rhinoconjunctivitis and to the previous occurrence of, for example, cradle cap, pruritus which is intensified by sweating, metal incompatibility or photophobia. Clinical signs such as dry skin, inflammation e.g. in the bend of the knee or elbow or on certain areas in the facial region are important indicators to atopy.

A particular challenge is the difficulty of diagnosis, e.g. in the case of infantile bronchial asthma. Often the clinical picture is classified as recurrent obstructive bronchitis for far too long, and the diagnosis of bronchial asthma is established too late. In addition to the medical history and the detection of early sensitization and/or concurrent atopic dermatitis, in recent years eosinophil cationic protein (ECP) has been employed for identifying children at risk (ECP>16 μg/l) of infantile bronchial asthma.

Furthermore, determination of ECP is also helpful for monitoring the effectiveness of antiinflammatory treatment. Apart from determination of ECP, measurement of baby lung function with methacholine provocation is a good means for confirming the diagnosis of bronchial asthma in doubtful cases even in very young children. There is no correlation between ECP and bronchial hyperreactivity, as these two methods detect different pathogenetic mechanisms of bronchial asthma. A clear diagnosis should be obtained early for all young children with obstructive symptoms (wheezing), in order to avoid the development of chronic bronchial asthma.

With regard to laboratory tests, for all diseases of the atopic classification, primarily the concentration of total IgE antibodies in the blood is determined. An elevated concentration is indicative of atopy or allergy.

Various immunological methods are used in the laboratory for determining total IgE. The results are stated in IU/ml (international units) or KU/l.

The concentration of total IgE is, for example, often determined by ELISA (enzyme linked immunosorbent assay). For this, e.g. in a so-called sandwich-ELISA, a support is coated with anti-human IgE antibodies of polyclonal origin, and nonspecific binding sites are blocked, e.g. with BSA (bovine serum albumin). The patient's serum, e.g. at 1:10 dilution, is brought into contact with the support, washed, and bound IgE is detected with secondary antibodies, namely anti-human IgE antibodies (in the case of a human patient) These antibodies are generally labeled, e.g. with an enzyme such as alkaline peroxidase or horseradish peroxidase, which catalyzes a color reaction that is easy to detect and quantify. The total IgE concentration can also be determined by blotting (Western blot or dot blot), RIA (radio immunosorbent assay) or by means of magnetic beads as supports and fluorescence-labeled secondary antibodies.

In allergy sufferers, the total IgE is often raised compared with non-allergic subjects, but there is an overlap in the distribution of the IgE values. As a guide:
With values of less than 20 IU/ml (or KU/l) an allergy is rather unlikely.
With values of more than 100 IU/ml an allergy is probable.
With values between 20 and 100 IU/ml, no clear decision can be made based on the total IgE value.

In view of these limitations, this test is to be regarded as providing guidance.

Values above 100 IU/ml may however, for example when the medical history is uncertain, indicate that the patient's complaints can possibly be attributed to an allergy or atopy.

Against this background, a person skilled in the art is therefore faced with the problem of devising supplementary methods for the diagnosis of atopic and allergic diseases and of autoimmune diseases, which increase the reliability of a diagnosis or even make a diagnosis possible.

This problem is solved by the subject-matter of the claims, in particular by the use of CD28 for the diagnosis of allergic diseases, atopic diseases and/or autoimmune diseases. CD28 can be used for testing a sample from a patient for the presence of anti-CD28 and autoantibodies, by contacting the sample with CD28, wherein binding of the autoantibodies to CD28 indicates the presence of an allergic disease, atopic disease and/or autoimmune disease.

In this context, the present invention also provides a method for the diagnosis of allergic diseases, atopic diseases and/or autoimmune diseases, in which a sample from a patient is tested for the presence of anti-CD28 autoantibodies, by contacting the samples with CD28, wherein binding of the autoantibodies to CD28 indicates the presence of an allergic disease, atopic disease and/or autoimmune disease.

CD28 is expressed by resting and activated T-cells as a 44-kDA membrane protein. CD28 plays an essential role in induction of T-cell-mediated immune responses. The activation of naive T-cells requires at least two receptor-mediated signals, which are mediated by antigen-presenting cells (APCs).

The first signal is antigen-specific and is mediated by the interaction between the major histocompatibility complex (MHC) and the T-cell receptor (TCR). However, this signal is not sufficient for the activation of naive T-cells on its own. There must additionally be binding between CD28 on the T-lymphocytes and the respective ligands on the APCs, namely CD80 (B 7-1) or CD86 (B7-2) (Appleman et al., Immunol Rev 2003, 192: 161-180; Sharpe et al., Nature Rev 2002, 2: 116-126). Then the cells begin to proliferate and to differentiate into effector cells. The molecule CTLA-4 is also expressed on T-lymphocytes and can bind to CD80 and CD86. In contrast to CD28, CTLA-4 inhibits the effector response of activated T-lymphocytes. If for example the regulation of the T-cells is disturbed by CD28 and CTLA-4, autoreactive T-cells can be stimulated, and play a central pathophysiological role in autoimmune diseases, among others.

Autoantibodies to surface molecules of T-lymphocytes have been found in various autoimmune diseases, as well as in infections and during blood transfusions (Osman et al., Clin Rheumatol 1994, 13: 21-27; Swaak, Lymphocytotoxic Antibodies. In: Peter J. B., Shoenfeld Y, editors. Autoantibodies. Amsterdam: Elsevier Science, 1996: 478). The appearance of these antibodies correlates in some diseases with the activity of the disease (Winfield et al., Clin Immunol 1992; 63: 13-16) and with functional disturbances of the leukocytes (Winfield et al., Arthritis Rheumatol 1975, 18: 587-594; Morimoto et al., J Clin Invest 1987, 79: 762-768; Tanaka et al., Arthritis Rheum, 1989, 32: 398-405; Sakane et al., J Clin Invest 1979, 63: 954-965; Wernet et al., J Exp Med 1973, 138: 1021-1026; Takeuchi et al., Scand J Immunol 1982, 16: 369-377).

To date, autoantibodies to CD45, β₂-microglobulin and to HLA-Class 1 molecules have been found in humans (Mimura et al., J Exp Med 1990, 172: 653-656; Czyzyk et al. Arthritis Rheum 1996, 39: 592-599; Revillard et al., J Immunol 1979, 122: 614-618; Proper et al., Clin Sci (Lond) 1991, 80: 87-93), and in animals, autoantibodies to CTLA-4 (Khatlani et al., J Immunother 2003, 26: 12-20). To date, autoantibodies to CD28 have not been described (Khatlani et al.; Matsui et al., J Immunol 1999, 162: 4328-4335).

Within the scope of this invention it was found, surprisingly, that the occurrence of CD28 autoantibodies is significantly associated with atopic diseases, allergic diseases and autoimmune diseases, e.g. with atopic dermatitis (odds ratio 25.31 [95% CI (confidence interval), 5.52-116.11]; (p<0.0001), allergic asthma and rhinoconjunctivitis allergica (odds ratio 10.78 [95% CI, 5.39-21.55]; p<0.0001) and autoimmune diseases such as scleroderma. All other diseases that were diagnosed in patients whose serum was analysed were not correlated with the occurrence of CD28 autoantibodies (FIG. 4, Table 2).

Basically it was found that the presence of CD28 autoantibodies tends to be correlated with younger age and female gender. In order to exclude other influences, e.g. serum IgE, in addition a multivariate logistic regression analysis was performed. In this way a possible influence of age, of gender or of serum IgE was excluded statistically as a cofactor (FIG. 5, Table 3).

Within the scope of this invention, a full-length CD28-molecule or a fragment thereof, which can be recognized by anti-CD28 autoantibodies, is designated as CD28. Preferably it is an extracellular fragment. In particular the extracellular fragment of CD28 comprises the amino acids occurring in full-length CD28 except for the intracellular moiety and the transmembrane region. Preferably the extracellular fragment has the sequence according to SEQ ID NO:2. This describes the sequence of a human extracellular fragment.

The sequence of full-length human CD28 is shown in SEQ ID NO:1. However, in addition to human CD28-molecules or fragments thereof, CD28-proteins or fragments thereof from other species also fall within the scope of this invention, for example from mouse, rat, rabbit, guinea pig, dog, cat, horse or cow.

The full-length CD28-molecule or the extracellular fragment thereof can be part of a fusion protein. Preferably the fusion protein further comprises glutathione-S-transferase or a histidine tag, which is particularly useful for purification of the recombinant protein. Basically CD28 can be purified from cells or can be produced by recombinant techniques. The fusion protein can include an Ig (immunoglobulin) moiety, though it is preferred if this is not contained in the fusion protein, to avoid difficulties with possible cross-reactivity of autoantibodies to the Ig moiety present in the patient's serum. CD28 can, however, be cleaved from CD28-Ig fusion molecules, e.g. with trypsin.

The patient's sample is preferably a blood sample or a serum sample. The method according to the invention is generally carried out in vitro.

Preferably CD28 is bound to a support. Said solid support can be, for example, an ELISA plate, a magnetic bead or a blot film, e.g. a nitrocellulose film. Supports, within the scope of the invention, are also cells that express CD28 on their surface naturally or by recombinant techniques. CD28 can be bound to the support directly, or, e.g. can be coupled to a support via antibodies, in particular antibodies to a tag linked to CD28, such as glutathione-S-transferase. These antibodies are not human antibodies, to prevent cross-reactions with secondary antibodies.

In a preferred embodiment of the invention, the binding of anti-CD28 autoantibodies to CD28 is investigated, by contacting the support with labeled anti-immunoglobulin antibodies to antibodies of the species to which the patient belongs, and detecting the labeled antibodies. The patient can, for example, be a human. In this case preferably human CD28 is used, and the anti-immunoglobulin antibodies are anti-human immunoglobulin antibodies.

Preferably the anti-immunoglobulin antibodies are specific to antibodies of the IgG isotype, though they can also be reactive to IgG and IgM and/or IgE.

Preferably the anti-immunoglobulin antibodies are labeled with an enzyme, for example alkaline phosphatase or horseradish peroxidase, biotin, a radioactive isotope or a fluorescent dye, e.g. fluorescein isothiocyanate (FITC) or phycoerythrin (PE).

The investigation can be carried out in a blot, e.g. a Dot blot or a Western blot, an ELISA (enzyme linked immunosorbent assay), an RIA (radioimmunoassay), a FACS (fluorescence activated cell sorting) analysis or also in a liquid-phase detection system. If the supports are cells, the subsequent investigation of binding is preferably performed by FACS or fluorescence microscopy.

In particular, the method according to the invention or the use of CD28 according to the invention can be used for the diagnosis of rhinoconjunctivitis allergica (hayfever) or allergic bronchial asthma. An especially high correlation between CD28 autoantibodies and disease is also found in atopic dermatitis. In the class of autoimmune diseases, by means of anti-CD28 autoantibodies it is possible to detect, in particular, scleroderma or lupus erythematodes, but also rheumatoid arthritis and dermatomyositis.

Anti-CD28 autoantibodies also occur in bullous autoimmune diseases of the skin (pemphigus vulgaris, bullous pemphigoid).

The detection of anti-CD28 antibodies can be used without supplementary diagnostic techniques, in particular laboratory tests, for the diagnosis of one of the stated diseases. However, combination with other criteria is preferred. In particular, the patient's medical record and the family medical history, and the clinical symptoms, continue of course to play a dominant role in diagnosis.

In the case of additional laboratory tests, in the atopic and allergic classification the determination of the concentration of total IgE in a sample from a patient, for example a blood sample or serum sample, is particularly important. Thus, a concentration of 100 IU/ml total IgE or more indicates the presence of an allergic disease and/or atopic disease. A concentration of 20-100 IU/ml total IgE does not provide a precise indication of the presence of such a disease (Sanz M L, Prieto I, Garcia B E, Oehling A. Diagnostic reliability considerations of specific IgE determination. J Invest Allergol Clin Immunol. 1996 May-June; 6(3):152-61). Especially in this case, a supplementary diagnosis with the aid of CD28 is sensible.

If asthma is suspected, combination of the method of diagnosis according to the invention with provocative tests, e.g. with methacholine, is sensible. Other tests, e.g. for the eosinophil cationic protein (Wolthers O D. Eosinophil granule proteins in the assessment of airway inflammation in pediatric bronchial asthma. Pediatr Allergy Immunol. 2003 August; 14(4):248-54), can be combined with the method according to the invention.

Within the scope of the present invention, a method is furthermore made available for the diagnosis of a special risk, of an especially severe disease or of a particular intensity of the disease, as it was established that there is positive correlation with the concentration of autoantibodies to CD28. An important parameter for the severity of an atopic disease is the serum IgE. For the patients investigated, a correlation was found between the level of serum IgE and the level of the titer of the anti-CD28 autoantibodies.

A kit is also provided for carrying out a method according to the invention. Preferably this kit is suitable for the diagnosis of allergic diseases and/or atopic diseases and comprises CD28 and labeled anti-immunoglobulin antibodies.

Preferably it comprises human CD28 and labeled anti-human immunoglobulin antibodies, in particular anti-human IgG antibodies.

In a preferred embodiment the kit according to the invention further comprises labeled anti-IgE antibodies and so is suitable for carrying out diagnostic tests for the ascertainment of allergic or atopic diseases both on the basis of determination of the concentration of total IgE and on the basis of detection of CD28 autoantibodies. The kit can further comprise unlabeled anti-IgE antibodies, so that the test for total IgE can be carried out as a sandwich-ELISA. The unlabeled antibodies can be polyclonal anti-IgE antibodies, and the labeled anti-IgE antibodies can be polyclonal or monoclonal. Alternatively the labeled and the unlabeled anti-IgE antibodies can each be monoclonal antibodies, which must, however, be directed against a different epitope.

The labeled antibodies contained in the kit can be labeled with an enzyme, e.g. alkaline phosphatase or horseradish peroxidase, biotin, a radioactive isotope or a fluorescent dye, e.g. FITC or PE.

### EXAMPLES

### Example 1

### Enzymatic Cleavage of the Recombinant CD28-Ig Fusion Protein with Trypsin and Detection of Autoantibodies to CD28 in the Immunoblot

The recombinant CD28-Ig fusion protein (R & D Systems Inc. Minneapolis, USA) was dissolved in PBS buffer (2.7 M NaCl, 54 mM KCl, 87 mM Na₂HPO₄, 30 mM KHPO₄, pH 7.4) at a concentration of 1 mg/ml and 100 μl of this solution was digested with 50 μl trypsin (10 mg/ml) at 37° C. for 15 minutes. At the end of the incubation time, 1.5 μl aprotinin (10 mg/ml) and 2.5 μl TLCK (Nα-p-tosyl-L-lysine-chloromethyl ketone, 20 mg/ml) were added, in order to inhibit the enzymatic activity of trypsin. The solution can be stored at −20° C. until further use.

The separation of proteins by gel electrophoresis (SDS-PAGE) is carried out according to the method of Lughtenberg et al. (Lughtenberg, B. (1975), FEBS Lett. 58, 254). The cleavage products were separated by SDS-gel electrophoresis (10% gel) with a 4% stacking gel (110 V, 150 minutes) in non-reducing conditions. Next the cleavage products were transferred at a current of 50 mA for 3 hours on PVDF (polyvinylidene fluoride) membranes (Segin-Blot, Biorad, Germany). Then the membranes were blocked with 5% skim-milk powder for 60 minutes at room temperature and washed three times with PBS.

The sensitivity and specificity of the immunblot were monitored with the following antibodies: monoclonal mouse anti-human CD28 antibodies (R&D Systems, Minneapolis, USA), diluted 1:5000 in PBS, biotinylated polyclonal mouse anti-human CD28 antibodies (R&D Systems, Minneapolis, USA), diluted 1:5000 in PBS, monoclonal mouse anti-human Fc antibodies (Dianova, Hamburg, Germany), diluted 1:10 000 in PBS, and polyclonal rabbit anti-human IgG antibodies (Sigma-Aldrich, Steinheim, Germany), diluted-1:3500 in PBS.

FIG. 1 shows that a cleavage product of the CD28-Ig fusion protein is only recognized unambiguously by the anti-CD28 antibodies, and not by antibodies to IgG or Fc.

For detecting the autoantibodies to CD28 in the serum, the PVDF membranes cut into strips were incubated in 1:10 diluted human serum for 1 hour on a swivel table at room temperature. Specific antisera to human Fc (Dianova, Hamburg) and human CD28 (R&D, Wiesbaden) served as controls. Then the blots were washed again three times and then incubated for 1 hour at room temperature with a secondary, AP-conjugated antibody to human IgG (from Serva, Heidelberg). The binding was visualized by an enzymatic color reaction (BCIP/NBT, 5-bromo-4-chloro-3-indolyl phosphate/p-nitroblue tetrazolium chloride).

FIG. 2 shows, as examples, the results for sera that contain autoantibodies to CD28 or are negative. By means of the immunoblot, specific IgG antibodies to CD28 can be detected in the serum.

### Example 2

### Association of Autoantibodies to CD28 with Different Diseases

Using the method, a total of 268 sera were tested and evaluated for the presence of autoantibodies to CD28. In this group, 72 sera were obtained from healthy test subjects, and 196 sera were from patients with various diseases. Written consent to the taking of blood samples for scientific purposes had been obtained from each patient.

In the group of healthy test subjects, CD28 autoantibodies were detected in 8/72 sera (11.1%). In the patient group, 53/196 sera (27.04%) were positive. Table 1 shows that the presence of CD28 autoantibodies tends to be correlated with younger age and female gender.

TABLE 1
Age and gender in relation to anti-CD28 autoantibodies

| | CD28+ | CD28− | p |
|---|---|---|---|
| Age (±SD) | 52.3 ± 18.8 | 58.7 ± 19.9 | 0.073 |
| [Years] | | | |
| Sex | | | |
| male | 19 | 69 | |
| female | 34 | 74 | 0.146 |

SD: Standard deviation

Univariate analysis shows that the occurrence of CD28 autoantibodies is associated highly significantly with atopic eczema (odds ratio, 25.31 [95% CI, 5.52-116.11]; p<0.0001), allergic asthma and rhinoconjunctivitis allergica (OR 10.78 [95% CI, 5.39-21.55]; p<0.0001) and with autoimmune diseases such as scleroderma, lupus erythematodes, rheumatoid arthritis, dermatomyositis or bullous autoimmune diseases (Table 2). All other diseases that were diagnosed in the patient group were not correlated with the occurrence of CD28 autoantibodies.

In order to exclude other factors, e.g. serum-IgE, in addition a multivariate logistic regression analysis was carried out. On this basis, possible influence of age, of gender or of IgE in the serum as cofactors could be ruled out statistically (Table 3).

TABLE 2
Association between diagnosis and anti-CD28 autoantibodies.

| | Total | anti-CD28+ | anti-CD28− | Odds-Ratio | 95% CI | p* |
|---|---|---|---|---|---|---|
| Healthy controls | 72 | 8 | 64 | — | — | — |
| Atopic dermatitis | 16 | 14 | 2 | 56.00 | 28.4-110.4 | <0.0001 |
| Allergic rhinitis/asthma | 54 | 31 | 23 | 10.78 | 5.39-21.55 | <0.0001 |
| Autoimmune diseases | 8 | 5 | 3 | 13.33 | 5.72-31.1 | <0.01 |
| Cutaneous lymphoma | 3 | 1 | 2 | 4.00 | 0.71-22.5 | n.s. |
| Non-melanoma skin cancer | 27 | 9 | 18 | 4.00 | 1.72-9.30 | n.s. |
| Leg ulcer | 53 | 13 | 40 | 2.60 | 1.16-5.82 | n.s. |
| Skin infections | 24 | 5 | 19 | 2.11 | 0.76-5.82 | n.s. |
| Other inflammatory skin diseases | 49 | 10 | 39 | 2.05 | 0.87-4.83 | n.s. |
| Psoriasis | 9 | 1 | 8 | 1.00 | 0.14-7.10 | n.s. |
| Malignant melanoma | 11 | 1 | 10 | 0.80 | 0.11-5.79 | n.s. |

95%-CI: 95 percent confidence interval;
n.s.: not significant;
p*: Bonferroni-corrected p-value with Fisher's exact test;
OR: odds ratio

TABLE 3
Logistic regression analysis of factors having an influence
on the occurrence of anti-CD28 autoantibodies

| Factor | OR | 95% CI | P |
|---|---|---|---|
| Age | 0.993 | 0.974-1.012 | 0.443 |
| Gender | 1.073 | 0.503-2.288 | 0.855 |
| IgE [kU/l] | 0.999 | 0.999-1.000 | 0.077 |
| Allergic rhinitis/asthma | 2.484 | 1.139-5.417 | 0.022 |
| Atopic dermatitis | 62.682 | 6.296-624.02 | 0.0004 |
| Autoimmune diseases | 8.909 | 1.572-50.48 | 0.014 |

### Example 3

### Functional Significance of the Autoantibodies to CD28

The functional significance of the autoantibodies to CD28 was investigated by means of a so-called mixed lymphocyte reaction (MLR).

First, protein G-beads (Dynal, Hamburg) were loaded with the CD28 fusion protein according to the instructions. The protein was bound irreversibly to the beads by crosslinking, and binding was measured by flow cytometry. Then serum pools were prepared from (1) three sera with anti-CD28 autoantibodies from patients with atopic eczema (AE+), (2) two sera without anti-CD28 autoantibodies from patients with atopic eczema (AEØ), (3) two sera with anti-CD28 autoantibodies from patients without atopic eczema (G+) and (4) from seven sera without anti-CD28 autoantibodies from patients without atopic eczema (GØ).

The pool sera were purified successively over the beads, so that the fractions with the antibodies to CD28 and to human Fc were obtained. The eluates were then tested by Western blotting for the presence of antibodies to CD28.

Next, a proliferation test was carried out with the eluates and the pool sera within the scope of an MLR. The cell lines were cultivated in RPMI 1640 medium+10% fetal calf serum (FCS). For the MLR, Jurkat cells (10⁴ cells/ml) were cultivated together with irradiated (dose: 30 gy) Raji cells (10⁴/ml). The eluates (50 μl) were added to various media. In some media, 4 μg CTLA-4-Ig (R&D Systems, Minneapolis, USA) was added for induction of anergy. After 2 days, 5-bromo-2′-deoxyuridine (BrdU) was added to the cultures and incubated for 5 hours (Neuber et al., Immunology 2003, 109: 24-31). Then the proliferation of the cells was measured by means of a color reaction.

The experiments showed (FIG. 3) that the eluates from sera with autoantibodies to CD28 greatly stimulated cell proliferation, whereas the sera without autoantibodies inhibited proliferation.

Stimulation of the CTLA-4 receptor on T-lymphocytes shifts the cells to an anergic state through inhibition of the stimulatory signals (Sharpe et al., Nature Rev 2002, 2: 116-126). Therefore the CTLA-4-Ig fusion protein was used as control in the proliferation experiments (Linsley et al., J Exp Med 1991, 174: 561-569). It was found that sera with autoantibodies to CD28 break through the anergic state and the T-cells can proliferate again.

### Example 4

### Production of a CD28-GST Fusion Protein

RNA Preparation from Human Whole Blood

First, RNA was prepared from human whole blood using the QIAamp RNA Blood Mini-Kit from the company Qiagen (Hilden, Catalog number: 52304).

RT-PCR for Production of cDNA

5 μg of the prepared RNA was transcribed into blood-cDNA with the Superscript kit (Invitrogen, Karlsruhe) with random hexamer primers (Catalog number: 53034).

Cloning of the CD28 cDNA

Then the cDNA region coding for the extracellular region (IgG-like domain, without transmembrane region and signal peptide) of this protein was amplified by a polymerase chain reaction (PCR). The reference used was the sequence NM_{—}006139 (SEQ ID NO: 3) available online from NCBI.

The amplified cDNA codes for the amino acid sequence:

(SEQ ID NO: 2)

PSIQVTGNKILVKQSPMLVAYDNAVNLSCKYSYNLFSREFRASLHKGLDS
AVEVCVVYGNYSQQLQVYSKTGFNCDGKLGNESVTFYLQNLYVNQTDIYF
CKIEVMYPPPYLDNEKSNGTIIHVKGKIILCPSPLF

The primers used were:

(SEQ ID NO: 4)

| | |
|---|---|
| Sense | 5′-AAAGAATTCCCTTCAATTCAAGTAACAGGAAAC-3′ |

(SEQ ID NO: 5)

| | |
|---|---|
| Antisense | 3′-AAACCCGGGAAATAGGGGACTTGGACAAAG-3′ |

The cDNA was integrated in the multiple cloning site of the vector pGEX-4T-1 (Amersham Biosciences, Freiburg, Catalog number: 27-4580-01) via cleavage sites for the restriction enzymes EcoRI and SmaI, which had already been integrated into the primers 5′ used for amplification (underlined in the primer sequence). For this, both the PCR-amplification product and the vector pGEX-4T-1 were first incubated with SmaI (New England Biolabs, Frankfurt A.M., Catalog number: #R0141S) and then with EcoRI (New England Biolabs, Catalog number #R0101S) in the reaction buffer NEB4 at 20° C. or 37° C. for two hours in each case. This was followed by purification of the cleaved DNA by means of the agarose gel DNA extraction kit from the company Roche (Basel, Catalog number: 1696505). The linearized vector was then dephosphorylated, for which the eluted DNA was incubated at 37° C. with 10 U alkaline phosphatase (Roche, Catalog number: 713023) after addition of the appropriate buffer. The restriction preparations were then separated in 1% agarose gel at 100 V. After ethidium-bromide staining, a band of approx. 400 bp could be visualized on a UV transilluminator and cut out. The cleaved cDNA was then eluted using the Roche agarose gel elution kit in 50 μl H₂O. Next, a ligation charge was prepared with T4-ligase (Invitrogen, Catalog number: E111-01), 100 ng vector and 200 ng cDNA fragment and incubated at 12° C. for several hours. This yields a construct in which the glutathione S-transferase is located in the 5′ direction from the CD28 region present in the reading frame.

Next, competent bacteria (XL1 Blue, HB101) were transformed with one fifth of the ligation charge. For this, a fifth of the ligation charge or 0.5 μg of a DNA preparation was added by pipette to 50 μl of competent bacteria thawed on ice, and incubated for 30 min on ice.

Then the bacterial preparations were heated for 5 min at 37° C. Then 950 μl of SOC medium was added and between 50 μl and 1 ml of the charge was streaked uniformly on LB-agar plates with 150 μg/ml ampicillin with a pipette, and incubated overnight at 37° C.

Minipreparation of Plasmid DNA from Bacteria

Individual colonies of transformed bacteria were inoculated with a corresponding antibiotic additive in 2.5 ml medium and incubated overnight. The bacteria were then centrifuged at 2400 g, the supernatant was drawn off and the pellet was resuspended in 200 μl STET (8% sucrose, 5% Triton X-100, 50 mM Tris-HCl, pH 8.0, 50 mM EDTA) with 1 μg/ml lysozyme on a shaker. The charge was then heated for 2 minutes at 95° C. and then centrifuged for 10 min at 16000 g. The sediment was removed with a toothpick and discarded. 10 μl of 5% CTAB solution (cetyl-trimethyl-ammonium bromide) was added to the supernatant, shaken briefly and the precipitate was sedimented at 16 000 g. The supernatant was drawn off and the pellet was dissolved in 300 μl of 1.2 M NaCl on a shaker. Then 750 μl of 100% ethanol was added, the charge was shaken vigorously and centrifuged for 10 min at 16000 g. The supernatant was drawn off, and the pellet was washed in 1 ml of 70% ethanol, centrifuged for 5 min at 16 000 g, and the supernatant was again discarded. The DNA pellet was dried and then taken up in 30 μl H₂O.

The correctness of the sequence of the inserted region was determined by sequencing.

### Preparation of the Recombinant CD28-GST Fusion Protein

Competent BL21-RILsuppl. Bacteria (a protease-deficient E. coli strain) were transformed with the cDNA construct and, after preincubation in SOC medium at 37° C. for 30 min, streaked on LB-agar plates with 150 μg/ml ampicillin. After incubation for 14 h, 30 ml of LB medium with ampicillin was inoculated with a colony.

This preliminary culture was incubated overnight at 37° C. in the shaker. It was then transferred to 500 ml LB medium with ampicillin and incubated at 37° C., shaking continuously, up to an optical density of 0.6-0.8 at 600 nm (approx. 1.5 h).

Immediately on reaching this density, protein expression was induced with 1 mM IPTG (isopropyl bD-thiogalactopyranoside, Biomol, Hamburg, Catalog number: 05684-1). After approx. 4 hours the bacteria were sedimented at 4000 g and 4° C. for 10 minutes, the culture supernatant was discarded and the pellet was resuspended in 5-10 ml ice-cold PBS. This suspension was sonicated 5 times, for 10 seconds each time (Branson Sonifier 250, stage 6) and then centrifuged for 20 minutes at 30 000 g. Affinity purification was then carried out via GST-tag. Glutathione-Sepharose 4B (Amersham Biosciences, Catalog number: 27-4574-01) was loaded to a bed volume of 1 ml in a gravity-driven polypropylene column and equilibrated in five times the volume of PBS.

Then the bacterial lysate was loaded and flow to the column was started again. The second passage was discarded and the column was washed with five to ten times the bed volume of PBS. Elution was carried out in 10 mM reduced glutathione, 50 mM Tris pH 7.5, 100 mM NaCl, 10% glycerol. Aliquots of the eluted protein were then obtained, and were stored at −80° C.

### Materials Used

| PBS | 137 mM NaCl |
|---|---|
| | 2.7 mM KCl |
| | 7.4 mM Na₂HPO₄ |
| | 1.5 mM KH₂PO₄ |
| STET | 8% sucrose |
| | 0.1% Triton X |
| | 50 mM EDTA |
| | 50 mM Tris pH 8 |
| TAEx50 | 2 M Tris base |
| | 5.71% glacial acetic acid |
| | 50 mM EDTA |
| TE | 20 mM Tris pH 7.5 |
| | 1 mM EDTA |
| Application buffer x 5 | 40% sucrose |
| (for nucleic acids) | 0.25% bromophenol blue |
| | 0.25% Xylene X in TE |
| SOC medium | 2% Bacto-trypton |
| | 0.5% Bacto yeast extract |
| | 10 mM NaCl |
| | 2.5 mM KCl |
| | 5 N NaOH to pH 7.0 |
| Autoclave and then cool to 60° C. | |
| | 10 mM MgCl₂ |
| | 10 mM glucose |
| Dyt medium | 1.6% Bacto-tryptone |
| | 1% Bacto yeast extract |
| | 100 mM NaCl |
| LB medium | 1% Bacto-tryptone |
| | 0.5% Bacto yeast extract |
| | 200 mM NaCl |
| adjust to pH 7.5 with NaOH | |
| Y-broth | LB medium |
| | 4 mM MgSO₄ |
| | 5 mM KCl |
| TFB 1 | 15% glycerol |
| | 10 mM CaCl₂ |
| | 30 mM potassium acetate |
| adjust to pH 5.8 with acetic acid | |
| | 100 mM RbCl |
| | 50 mM MnCl₂ |
| TFB 2 | 15% glycerol |
| | 10 mM MOPS |
| | 75 mM CaCl₂ |
| | 10 mM RbCl |
| LB medium | 1% Bacto-tryptone |
| | 0.5% Bacto yeast extract |
| | 200 mM NaCl |

adjust to pH 7.5 with NaOH

### Production of Competent Bacteria

XL1blue bacteria were streaked on an LB plate and incubated overnight at 37° C. In the morning a few colonies were each inoculated with 2 ml Y-broth and incubated for 2 hours at 37° C. in the shaker. Then these preliminary cultures were poured into 500 ml Y-broth and incubated to an OD at 600 nm of 0.3-0.35. Then the culture was distributed in two 50-ml polypropylene tubes, kept on ice for a short time, and sedimented at 4° C. and 2000 g. The supernatant was decanted, the pellet was resuspended, in each case in 15 ml, in TFB 1 (15% glycerol, 10 mM CaCl₂, 30 mM potassium acetate, adjusted to pH 5.8 with acetic acid, 100 mM RbCl, 50 mM MnCl₂) and kept on ice for 60-90 minutes.

Then it was sedimented again at 2000 g, the supernatant was decanted and the pellet was resuspended in each case in 2 ml TFB 2 (15% glycerol, 10 mM MOPS, 75 mM CaCl₂, 10 mM RbCl). The bacteria were then divided into 200 μl aliquots and immediately shock-frozen in liquid nitrogen and then stored at −80° C.

### Example 5

### ELISA for Detecting Specific Antibodies to the Extracellular Domain of Human CD28

The wells of microtiter plates (Maxisorp, Nunc) were coated with 100 μl monoclonal mouse anti-glutathione-S-transferase (GST) antibodies (specific to GST from Schistosoma japonicum, prediluted 1:2000 in PBS). After an incubation time of 1 hour at room temperature, the wells were washed 3 times with PBS+0.1% Tween 20.

Then blocking was carried out with 250 μl PBS with 1% skim-milk powder and 0.1% Tween 20 for 1 hour at room temperature, followed by washing again, 3 times with PBS+0.1% Tween 20.

In each case 100 μl of 1:2 prediluted CD28-GST antigen (PBS+0.1% Tween 20) was pipetted into the wells of columns 1 and 2, the wells of the third and fourth columns each received 100 μl PBS+0.1% Tween 20 only. The other columns of the microtiter plate are prepared similarly.

After one hour at room temperature, three more washing steps are carried out. Then the sera from the patients and the positive control serum, a polyclonal rabbit anti-human CD28 antibody (Santa Cruz, Heidelberg), concentration 1 μg/ml, are prediluted 1:200 in PBS+0.1% Tween 20.

The blank value contained the antigen, but no control serum, the negative control contained the control serum, but no antigen and the blank contained neither control serum nor antigen. Each patient serum was measured in double determinations against antigen and—to exclude nonspecific reactions—against PBS+0.1%/Tween 20.

Then the microtiter plate was incubated for 1 h at room temperature and then washed three times. In the next step, the secondary antibody was added. In the wells with the control serum (B 1-4) in each case 100 μl of an anti-rabbit IgG antibody was used (Fc-specific; Sigma, Munich) and for the patient sera, in each case 100 μl of an anti-human IgG antibody (Fc-specific; Sigma, Munich) was used. Both antibodies were prediluted 1:5000 and labeled with alkaline phosphatase. The incubation time was 60 minutes at room temperature.

Then after washing thoroughly five times, 100 μl of the p-nitrophenyl substrate solution (pNPP substrate tablet set, Sigma, Munich) was added to each well, and incubated in the dark for sixty minutes at room temperature. Color development was measured at 405 nm after 30 and after 60 minutes.

The result for each serum was calculated from the following formula:

$$Anti \text{-} CD 28 = \frac{{OD}_{Serum} [ 60 min ] - {OD}_{LW}}{{OD}_{LW}}$$

The quotient was calculated for all the sera tested.

The limit value was calculated with the 72 sera from the healthy test subjects. It was found that 95% of all the calculated quotients of the healthy test subjects were below 9, so that values>10 were assessed as positive, i.e. they contained autoantibodies to CD28.

In addition, sera which definitely did not contain autoantibodies to CD28 and sera that definitely contained CD28 autoantibodies were tested in a dilution series. It was found that sera that contained the CD28 autoantibodies also still showed an unambiguously higher OD at a dilution of 1:700 than the negative sera (FIG. 4).

Cross-reacting antibodies to GST in the patient sera could be ruled out. For this purpose, 32 sera were tested, and no serum was found to contain antibodies to the GST used in this ELISA.

For all patients with an atopic disease, the IgE values were present in the serum. The concentration of IgE is a parameter for the degree of severity of an atopic disease. The titers for autoantibodies to CD28 correlate, for these patients, significantly with the level of the serum IgE titer. Hence it can be concluded that the level of the anti-CD28 autoantibody titers also correlates with the severity of the atopy.

### FIGURES

FIG. 1:

Cleavage products after enzymatic digestion of the CD28-Ig fusion protein with trypsin.

Column 1: CD28 labeled with mouse anti-human CD28 moAb (monoclonal antibody). Column 2: CD28 labeled with a biotinylated polyclonal mouse anti-human CD28 Ab. Some smaller fragments of the CD28 fusion protein are detected with this polyclonal Ab, but not with the monoclonal antibody. Columns 3-5: Detection of split products of the Ig-part with rabbit anti-human IgG Ab (column 3) as well as with goat anti-human IgG Ab (column 4) and with mouse anti-human Fc Ab (column 5).

FIG. 2:

Immunoblots of 4 patients with atopic eczema (AD) and autoantibodies to CD28 as well as one patient without autoantibodies. As an example for CD28 antibodies in autoimmune diseases (AI), the result with the serum of a patient with sclerodermia is shown. In the patients shown here having epidermolysis bullosa acquisita (AI) and psoriasis vulgaris (PS), no autoantibodies to CD28 could be detected.

FIG. 3:

MLR with irradiated Raji cells and vital Jurkat cells. Proliferation was measured by the integration of BrdU after two days of culture. Results are shown as % stimulation of spontaneous proliferation (control). CTLA4-Ig inhibits proliferation, while eluates with CD28 autoantibodies (CD28 auto-Ab) significantly stimulate T cell proliferation. Costimulation with CTLA4-Ig and autoantibodies to CD28 showed a significantly decreased inhibition of Jurkat cell proliferation. **P<0.01, significant in comparison to the control.

FIG. 4:

Analysis of sera for anti-CD28 autoantibodies by ELISA. The OD of sera that definitely comprise anti-CD28 autoantibodies, even in high dilutions, is still significantly higher than for sera that are unambiguously negative for anti-CD28 autoantibodies.

FIG. 5:

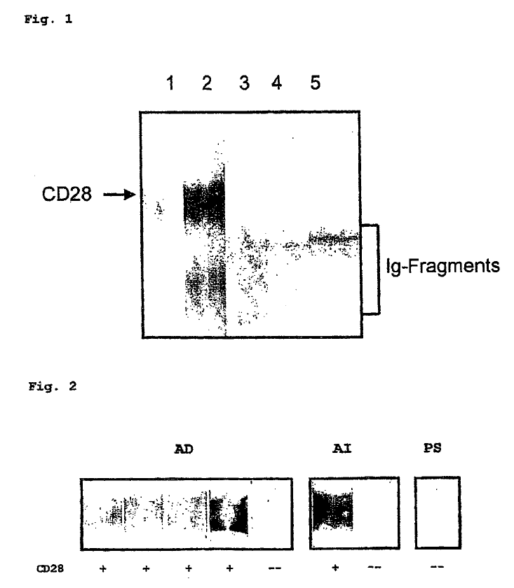

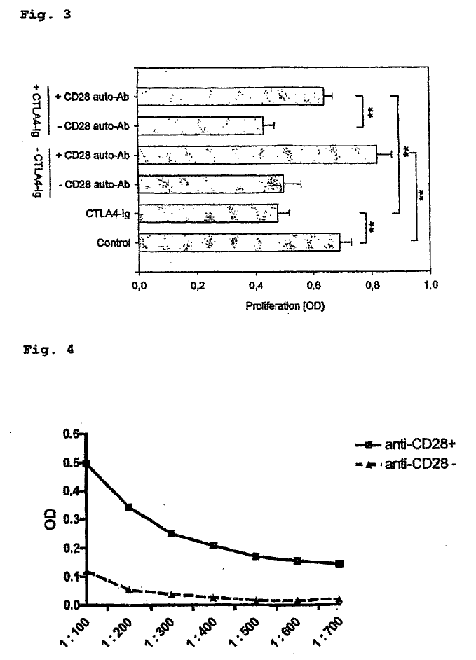

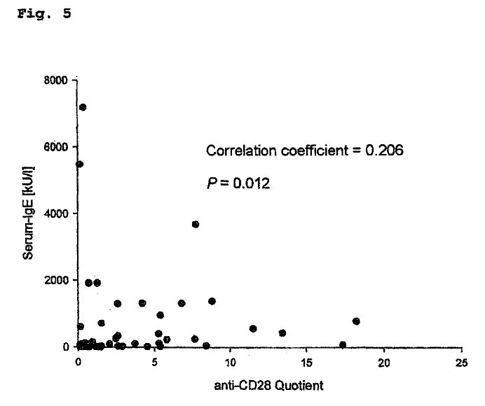

Presentation of the correlation between anti-CD28 autoantibodies and serum IgE in patients with atopy or atopical eczema, respectively. The coefficient of correlation is 0.206 and the level of significance is 0.012.

1-25. (canceled)

26. A method for the diagnosis of allergic diseases, atopic diseases and/or autoimmune diseases comprising providing a sample from a patient with a CD28 molecule or fragment thereof; analyzing for the presence of anti-CD28 autoantibodies; and correlating binding of said autoantibodies to said CD28 molecule or fragment thereof with the presence of an allergic disease, atopic disease and/or autoimmune disease.

27. The method of claim 26, wherein said CD28 molecule or fragment thereof comprises a full length CD28 molecule.

28. The method of claim 26, wherein said CD28 molecule or fragment thereof comprises an extracellular fragment of said CD28 molecule.

29. The method of claim 28, wherein said extracellular fragment has the sequence set forth in SEQ ID NO: 2.

30. The method of claim 26, wherein said CD28 molecule or fragment thereof comprise a fusion protein.

31. The method of claim 26, wherein said fusion protein further comprises glutathion-S-transferase.

32. The method of claim 26, wherein said sample is a blood sample or serum sample.

33. The method of claim 26, wherein said CD28 molecule or fragment thereof is bound to a support.

34. The method of claim 33, said analyzing comprises contacting said support with a labelled anti-immunoglobulin antibody to an antibody of the species to which the patient belongs, and detecting said labelled antibody.

35. The method of claim 34, wherein said patient is human and said anti-immunoglobulin antibody is an anti-human immunoglobulin antibody.

36. The method of claim 35, wherein said anti-immunoglobulin antibody is labelled with an enzyme, biotin, a radioactive isotope or a fluorescent dye.

37. The method of claim 36, wherein said analyzing is carried out in a blot, an ELISA, an RIA, an FACS analysis or a liquid phase detection system.

38. The method of claim 26, wherein said allergic disease is rhinoconjunctivitis allergica or allergic asthma bronchiale.

39. The method of claim 26, wherein said atopic disease is an atopic dermatitis.

40. The method of claim 26, wherein said autoimmune disease is sclerodermia, lupus erythematodes, rheumatoid arthritis, dermatomyositis or a bullous autoimmune disease.

41. The method of claim 26, further comprising determining the concentration of total IgE in said sample, and correlating a concentration of 100 IU/ml or more total IgE with the presence of an allergic disease and/or atopic disease.

42. The method of claim 26, further correlating a high concentration of autoantibodies to CD28 with a special risk, an especially severe disease or an especially intense disease.

43. A kit for the diagnosis of allergic diseases and/or atopic diseases comprising a CD28 molecule or fragment thereof and labelled anti-immunoglobulin antibodies.

44. The kit of claim 43, wherein said CD28 molecule or fragment thereof is a full-length CD28 molecule.

45. The kit of claim 43, wherein said CD28 molecule or fragment thereof is an extracellular fragment of said CD28 molecule or fragment thereof.

46. The kit of claim 45, wherein said extracellular fragment of a CD28 molecule or fragment thereof has the sequence as set forth in SEQ ID NO: 2.

47. The kit of claim 43, wherein said CD28 molecule or fragment thereof comprises a fusion protein.

48. The kit of claim 47, wherein said fusion protein further comprises glutathion-5-transferase.

49. The kit of claim 43, further comprising a labelled anti-IgE antibody.

50. The kit of claim 43, wherein said labelled antibodies are labelled with an enzyme, biotin, a radioactive isotope or a fluorescent dye.
